(19) United States
(12) Patent Application Publication
(10) Pub. No.: US 2011/0213317 A1
(43) Pub. Date: Sep. 1, 2011

(54) CANNULA FOR INTRAOCULAR SURGERY

(76) Inventors: David E-bin Chen, Fremont, CA (US); Erik William Peterson, Walnut Creek, CA (US)

(21) Appl. No.: 13/037,957

(22) Filed: Mar. 1, 2011

Related U.S. Application Data
(60) Provisional application No. 61/309,151, filed on Mar. 1, 2010.

(51) Int. Cl. A61M 25/00

(52) U.S. Cl. 604/264

(57) ABSTRACT

A cannula for intraocular surgery including a cup, a tube including a wall and having a tube outer diameter, the tube extending from the cup to a distal end and defining a longitudinal axis. In one implementation, the distal end includes a first portion and a second portion, the first portion having a tip and being narrower than the second portion. A gripping section is formed in the wall. The gripping section has a diameter that is less than the tube outer diameter. The gripping section allows the sclera to deform into the reduced diameter in order to increase a retention force of the cannula in the eye while minimizing damage to the tissue of the eye.

### CROSS-REFERENCE TO RELATED APPLICATIONS

This application claims priority to co-pending U.S. Provisional Patent Application No. 61/309,151 filed Mar. 1, 2010, the entire content of which is incorporated herein by reference.

### FIELD OF THE INVENTION

The present invention relates to cannulas for use in surgery, more specifically, the present invention relates to cannulas for intraocular surgery.

### BACKGROUND OF THE INVENTION

A trocar-cannula is a surgical instrument. It can be used to drain fluid from a body cavity, introduce fluids into the body cavity, and insert a tool into the body cavity to perform surgical functions. The trocar-cannula is comprised of two principal parts: (1) a cannula including a hollow tube and (2) a puncturing member referred to as an obturator or trocar. The trocar is passed through the hollow tube of the cannula, and is used to create an incision in the eye. The cannula is then inserted through the incision into the body cavity by sliding the cannula along the trocar.

A trocar-cannula may be used in cardiovascular surgery, laproscopic surgery, arthroscopic surgery, and intraocular surgery. However, the construction of the trocar-cannula is generally different for each application. For example, an arthroscopic trocar-cannula is usually too large for intraocular surgery. Furthermore, it may contain projections on the exterior surface of the lumen to prevent the cannula from inadvertently disengaging the joint. These projections could cause tissue damage if applied to the eye or other parts of the body.

In intraocular surgery, a trocar-cannula is often used to obtain access to the posterior-segment of the eye (the area behind the lens). Although surgical instruments can be introduced directly through incisions in the sclera of the eye, some surgeons introduce instruments through a cannula to reduce damage to the incision and nearby tissues.

### SUMMARY OF THE INVENTION

In one embodiment, the invention provides a cannula for intraocular surgery including a cup having an open first end and a second end and a hollow tube extending from the second end of the cup. The hollow tube includes a wall and a depressed gripping section for retaining the sclera of an eye by allowing the sclera to deform into the depressed gripping section.

In another embodiment, the invention provides a cannula for intraocular surgery, including a cup and tube. The tube includes a wall and has a tube diameter. The tube extends from the cup to a distal end and defines a longitudinal axis. The distal end includes a first portion and a second portion. The first portion includes a chamfered tip and is narrower than the second portion. A gripping section is formed in the wall. The gripping section has a diameter that is less than the tube diameter.

In another embodiment, the invention provides a cannula for intraocular surgery including a cup and a hollow tube. The cup has an open first end and a second end. The hollow tube extends from the second end of the cup to a distal end and defines a longitudinal axis. The distal end includes a first portion and a second portion. The first portion includes a tip and is narrower than the second portion.

Other aspects of the invention will become apparent by consideration of the detailed description and accompanying drawings.

### BRIEF DESCRIPTION OF THE DRAWINGS

FIG. 1 is a perspective view of a cannula.

FIG. 2 is a side view of the cannula shown in FIG. 1.

FIG. 2A is a sectional, side view of the cannula shown in FIG. 1 inserted into an incision in the sclera of an eye.

FIG. 3 is a perspective view of another cannula.

FIG. 4 is side view of the cannula shown in FIG. 3.

FIG. 5 is a perspective view of another cannula.

FIG. 6 is sectional side view of the cannula shown in FIG. 5.

FIG. 7 is a perspective view of another cannula.

FIG. 8 is a perspective view of another cannula.

FIG. 9 is sectional, side view of the cannula shown in FIG. 8.

FIG. 10 is a partial view of the cannula shown in FIG. 9.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of illustration and description of one or more examples of the invention and should not be regarded as limiting. It is possible that the invention could be embodied in forms not specifically described herein.

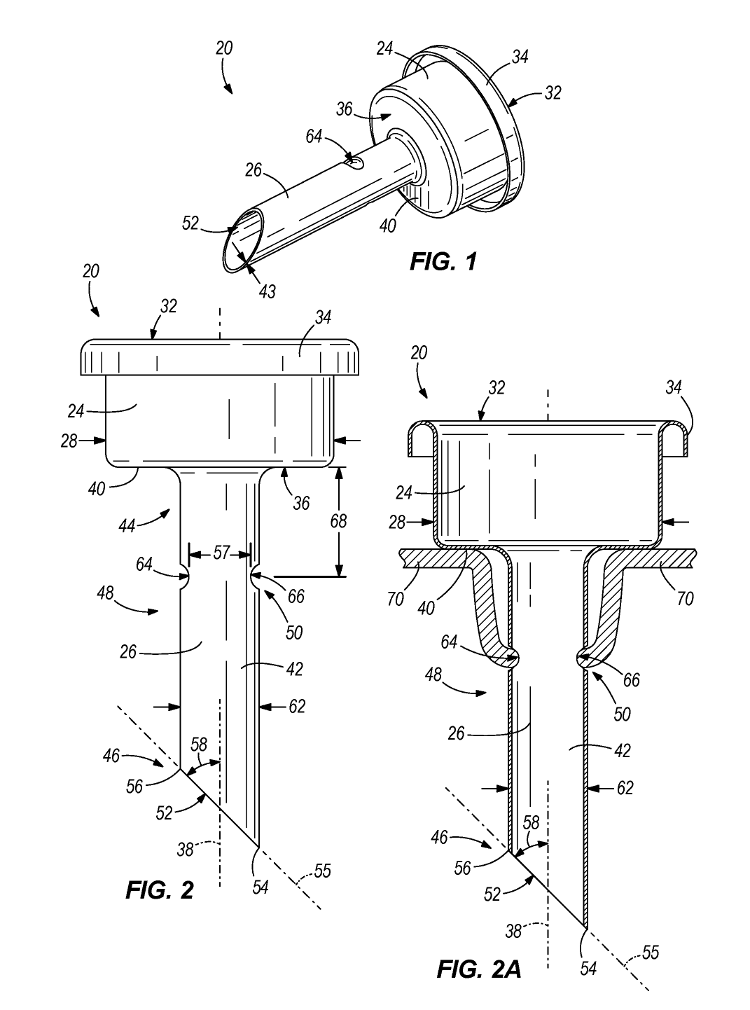

FIGS. 1 and 2 illustrate a cannula 20 including a body portion or cup 24 and a hollow tube 26. The cup 24 is generally cylindrical having an outer diameter 28 and including an open first end 32 about which is disposed a rolled lip 34. The hollow tube 26 extends from a second end 36 of the cup 24. A longitudinal axis 38 extends through the center of the cup 24 and the hollow tube 26. Preferably, the cannula 20 is constructed from a continuous piece of deep-drawn stainless steel. In some embodiments, the cannula 20 also includes an elastomeric self-sealing valve (not shown) that snaps over the rolled lip 34 of the cup 24 to cover the open first end 32 of the cup 24. Alternatively, the cup 24 is other shapes.

The hollow tube 26 is coaxial with the axis 38 and includes a generally cylindrical wall 42 having a wall thickness 43. The hollow tube 26 also includes a proximal end or first end 44 located at the second end 36 of the cup 24, a distal end or second end 46 opposite the first end 44, and an intermediate portion 48 disposed between the first end 44 and the second end 46. The intersection of the first end 44 of the hollow tube 26 and the second end 36 of the cup 24 creates a shoulder 40. The second end 46 includes a surface 52 that defines a tip 54 and a bevel termination 56. The surface 52 is included in a plane 55. The plane 55 is angled at an acute angle 58, measured relative to the axis 38. The first end 44, second end 46, and intermediate portion 48 have an outer diameter 62 that is less than the body outer diameter 28. The intermediate portion 48 includes a gripping section 50 with a gripping diameter 57 less than the outer diameter 62 of the hollow tube 26 so that the gripping section 50 is depressed relative to the intermediate portion 48. The gripping section 50 includes notches 64, 66 formed in the wall 42. In the illustrated embodiment, notch 64 is positioned opposite from notch 66 on the hollow tube 26 and the notches 64, 66 define the gripping diameter 57. The inner diameter of the hollow tube 26 remains substantially constant to allow surgical instruments to be inserted through the cannula 20 and into an eye. The notches 64, 66 are semicircular cutouts (when viewed from the side as shown in FIG. 2) that extend through the wall 42 of the hollow tube 26. The mid-point of the notches 64, 66 is positioned a notch distance 68 from the shoulder 40. Preferably, the notch distance 68 is about 0.04 millimeters (0.001575 inch).

In the illustrated embodiment, the notches 64, 66 extend through the wall 42 of the hollow tube 26. In other embodiments, the notches 64, 66 do not extend through the wall 42. Alternatively, the notches 64, 66 are portions of a single groove that extends around the circumference of the hollow tube 26. Other embodiments can have one or more notches of the same or different notch shape positioned on a different area of the hollow tube 26. Wire electrical discharge machining (or wire EDM) is particularly well suited to manufacture a cannula 20 with notch 64 positioned opposite from notch 66. Alternatively, the notches 64, 66 can be formed by driving a cylindrical grinding or cutting tool into the wall 42 of the hollow tube 26 in a direction perpendicular to the axis 38. The notches 64, 66 can be formed by operations including stamping, machining, drilling, or grinding. The notches 64, 66 can additionally be formed by boring a hole through both sides of the hollow tube 26. In other embodiments the notches 64, 66 can additionally be formed at the same time the hollow tube 26 is formed by injection molding, stamping, or other processes. In still other embodiments, the cannula 20 can be formed from other materials suitable for use in surgery including plastic. Alternatively, the notches 64, 66 are arranged in more rows than the two rows shown in the illustrated embodiment. The rows can be evenly spaced or unevenly spaced with respect to one another. For example, the notches 64, 66 could be arranged in three rows separated by 120 regress rather than two rows separated by 180 degrees. Selection among the illustrated embodiments and the alternative gripping sections and notch arrangements described above will depend on manufacturing technique, facilities, and expertise available to a manufacturer seeking to produce a cannula 20. Alternatively, the cup 24 is not included. When the cup 24 is not included a shoulder can be formed extending outward from the hollow tube 26 to provide a positioning surface to control the depth of insertion of the hollow tube 26 into the eye.

As shown in FIG. 2A, in use, the cannula 20 is first positioned on a trocar (not shown) by inserting the trocar through the cup 24 and the hollow tube 26. The trocar is then pressed into the sclera 70 of the eye. Next, the second end 46 of the hollow tube 26 of the cannula 20 is inserted into the incision created by the trocar. When the hollow tube 26 of the cannula 20 is inserted into the incision, the shoulder 40 at the second end 36 of the cup 24 creates a resting surface for the cannula 20 on the sclera 70. The notches 64, 66 of the gripping section 50 allow the sclera 70 to deform into the reduced diameter 57 defined by the notches 64, 66 in order to increase a retention force of the cannula 20 in the eye while minimizing damage to the tissue of the eye.

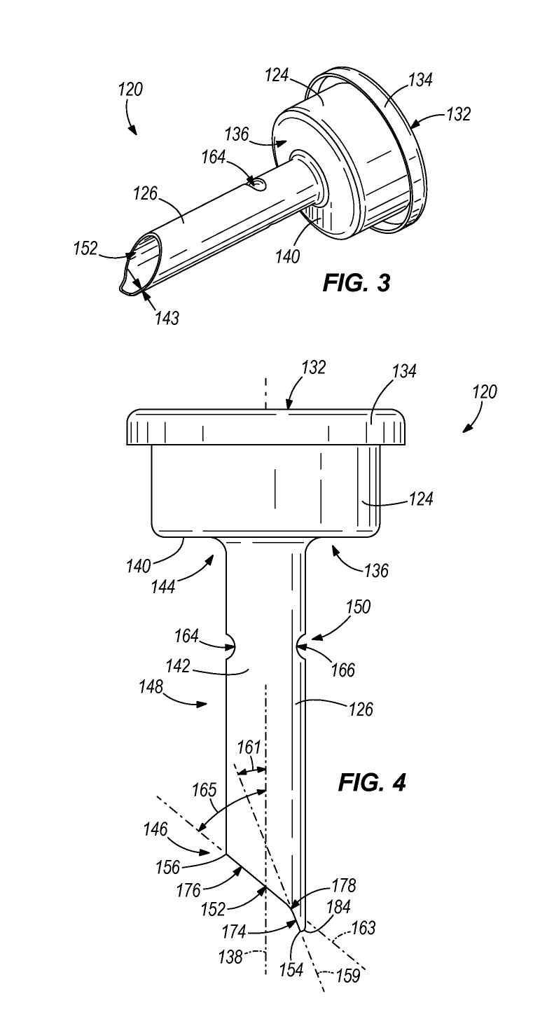

An alternative cannula 120 similar to cannula 20 is illustrated in FIGS. 3-4. Components similar to those of the cannula 20 described above are numbered in a similar fashion plus one hundred. The surface 152 includes a first portion 174 and a second portion 176. The first potion includes the tip 154. The first portion 174 is relatively narrow or pointy when compared to the second portion 176 to allow the first portion 174 to more easily locate and slide into the incision in the sclera. The first portion 174 is included in a plane 159 angled at an acute angle 161 relative to the axis 138. The second portion 176 is included in a plane 163 angled at an acute angle 165 relative to the axis 138. The angle 165 is greater than the angle 161. Preferably, the angle 161 is approximately 20 degrees and the angle 165 is approximately 50 degrees. A radius 178 defines the transition between the first portion 174 and the second portion 176. In the illustrated embodiment, the radius 178 is about 0.010 millimeter (0.0003937 inch). The first portion 174 allows the hollow tube 126 of the cannula 120 to be inserted into the incision in the eye with less force and difficulty than with known cannulas with beveled distal ends. The second portion 176 maintains strength in the second end 146 to prevent deformation or crushing of the hollow tube 126. Alternatively, additional portions, a curved surface having a changing radius, or a curvilinear surface could be used to form the surface 152 so that the first portion 174 is relatively narrow or pointy when compared to the second portion 176 or the remainder of the surface 152.

A chamfer 184 is formed in the wall 142 at the tip 154 to form a chamfered tip so that the tip 154 of the surface 152 is adjacent the trocar during insertion into the eye. The chamfer 184 displaces the sclera 70 of the eye slightly outward from the tip 154 to guide the sclera 70 along the hollow tube 126 to aid the sclera 70 in deforming or relaxing into notches 164, 166 when the cannula 120 is inserted into the eye. This relaxation helps increase the retention force of the cannula 120 in the eye while minimizing damage to the surrounding tissue.

The cannula 120 is used in a manner similar to the cannula 20. However, the first portion 174 and the chamfer 184 allow the cannula 120 to be inserted into the incision in the eye with reduced force and difficulty, while also reducing stress and damage to the eye, when compared to the cannula 20.

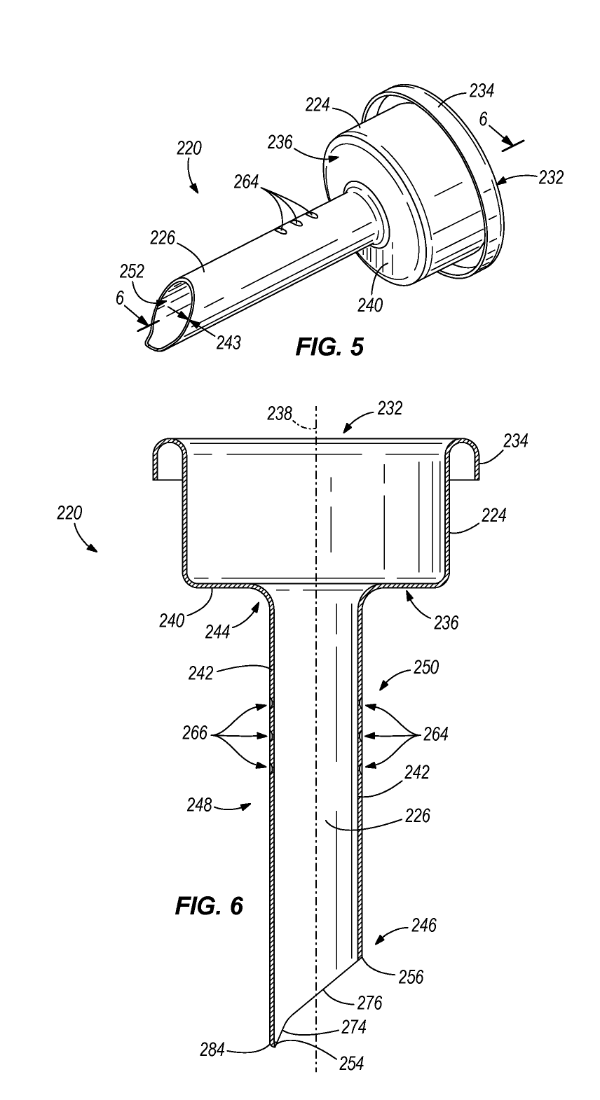

An alternative cannula 220 similar to cannula 120 is illustrated in FIGS. 5-6. Components similar to those of the cannula 120 described above are numbered in a similar fashion plus one hundred. Cannula 220 includes a gripping section 250 having three pairs of notches formed in the wall 242. Each pair of notches includes a first notch 264 and a second notch 266. In the illustrated embodiment, each first notch 264 is positioned opposite from the corresponding second notch 266. The notches 264, 266 extend into, but not through, the wall 242. Alternatively, more or fewer pairs of notches 264, 266 can be formed in the wall 242. The size and shape of the notches 264, 266 can vary. As shown in FIG. 6, the notches 264, 266 are semicircular. The radius of the semicircular notches 264, 266 can vary. In some embodiments, the gripping section 250 can be formed by pressing a die into the outside of the hollow tube 226 (with a mandrel inserted inside of the tube 226 to prevent collapse) to easily produce a multitude of shallow notches 264, 266 with the displaced material of the tube 226 forming raised edges adjacent to the notches 264, 266.

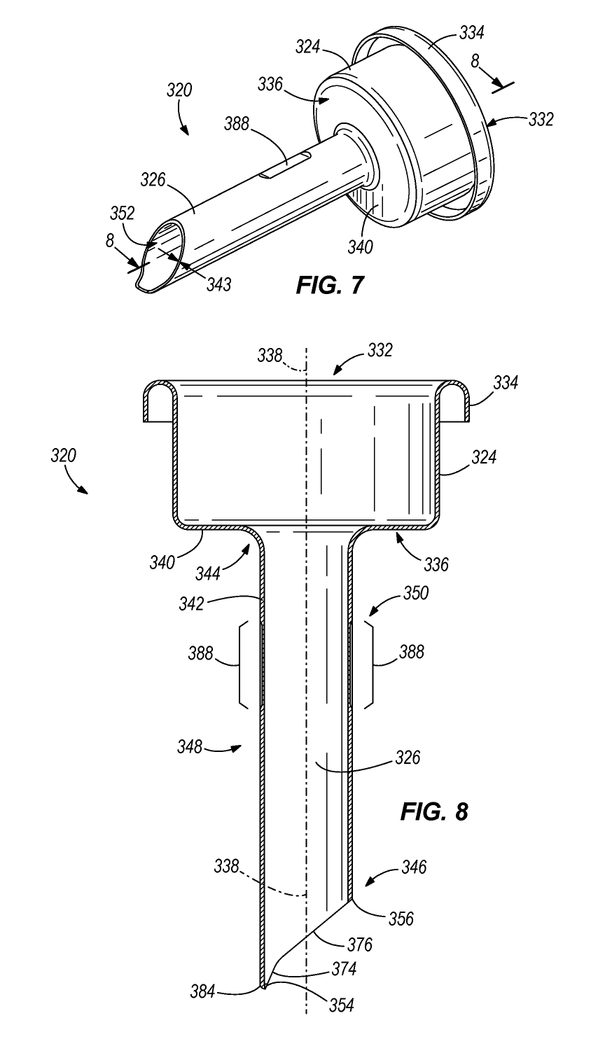

An alternative cannula 320 similar to cannula 120 is illustrated in FIGS. 7-8. Components similar to those of the cannula 120 described above are numbered in a similar fashion plus two hundred. Cannula 320 includes a gripping section 350 having two rectangular notches 388 positioned opposite one another. The rectangular notches 388 extend into, but not through, the wall 342 of the hollow tube 326. The dimensions of the rectangular notches 388 can vary. The edges formed in the wall 342 by the rectangular notches 388 can be straight, curved, or angled.

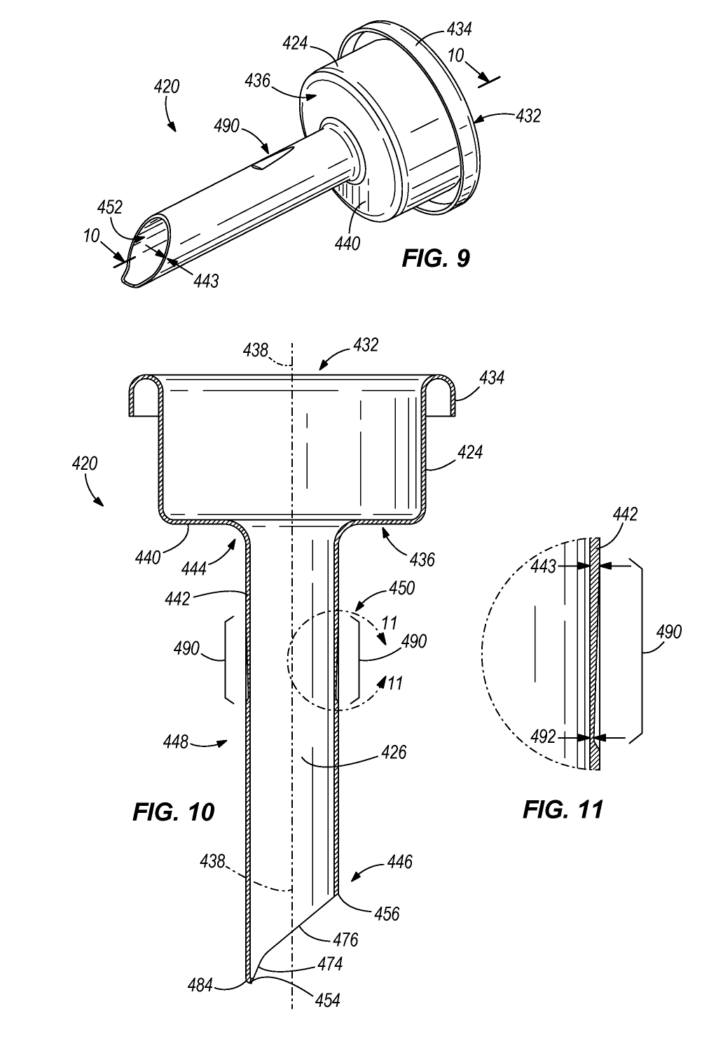

An alternative cannula 420 similar to cannula 320 is illustrated in FIGS. 9-11. Components similar to those of the cannula 320 described above are numbered in a similar fashion plus one hundred. Cannula 420 includes a gripping section 450 having two ramped notches 490 positioned opposite one another. The ramped notches 490 extend into, but not through, the wall 442. Each ramped notch 490 is formed by a reduction in the wall 442 from a first thickness 443 to a second thickness 492.

Variations in the embodiments of FIGS. 3-11 are similar to the variations of the embodiments in FIGS. 1-2A and include the notches of the gripping section extending through the wall of the hollow tube. In other embodiments there may be only one notch or multiple notches. The notches can be of various shapes. Other embodiments can have one or more notches of the same or different notch shape positioned on different areas of the hollow tube. The notches may be formed by operations including stamping, machining, drilling, grinding. The notches can additionally be formed by boring a hole through both sides of the hollow tube. In other embodiments the notches can be formed at the same time the hollow tube is formed by injection molding, stamping, or another process. In another embodiment, the gripping section can have very shallow continuous notches comprising a texturing of the cannula. In still other embodiments, the cannula can be formed from other materials including plastic. Alternatively, the notches are portions of a single groove that extends around the circumference of the hollow tube. Alternatively, the notches are arranged in more rows than the two rows shown in the illustrated embodiment. The rows can be evenly spaced or unevenly spaced with respect to one another. For example, the notches could be arranged in three rows separated by 120 regress rather than two rows separated by 180 degrees.

Various features and advantages of the invention are set forth in the following claims.

What is claimed is:

1. A cannula for intraocular surgery comprising:
a cup having an open first end and a second end; and
a hollow tube extending from the second end of the cup and including a wall and a depressed gripping section for retaining the sclera of an eye by allowing the sclera to deform into the depressed gripping section.

2. The cannula of claim 1 wherein the depressed gripping section includes a first notch formed in the wall and a second notch formed in the wall.

3. The cannula of claim 2 wherein the first notch is opposite from the second notch.

4. The cannula of claim 2 wherein the first notch extends through the wall and the second notch extends through the wall.

5. The cannula of claim 2 further comprising:
a third notch formed in the wall; and
a fourth notch formed in the wall.

6. The cannula of claim 1 wherein the tube extends from the cup to a distal end and the tube defines a longitudinal axis; and
wherein the distal end includes a first portion and a second portion, the first portion including a tip and being narrower than the second portion.

7. The cannula of claim 6 wherein the first portion defines a first plane that is acutely angled at a first angle relative to the longitudinal axis and the second portion defines a second plane that is acutely angled at a second angle relative to the longitudinal axis and the second angle is greater than the first angle.

8. The cannula of claim 1 wherein the tube extends from the cup to a distal end including a chamfered tip.

9. The cannula of claim 7 wherein the tip is chamfered.

10. The cannula of claim 2 wherein the tube extends from the cup to a distal end and the tube defines a longitudinal axis; and
wherein the distal end includes a first portion and a section portion, the first portion including a tip and being narrower than the second portion.

11. The cannula of claim 10 wherein the first portion defines a first plane that is acutely angled at a first angle relative to the longitudinal axis and the second portion defines a second plane that is acutely angled at a second angle relative to the longitudinal axis and the second angle is greater than the first angle.

12. The cannula of claim 10 wherein the tip is chamfered.

13. The cannula of claim 11 wherein the tip is chamfered.

14. The cannula of claim 5 wherein the tube extends from the cup to a distal end and the tube defines a longitudinal axis; and
wherein the distal end includes a first portion and a section portion, the first portion including a tip and being narrower than the second portion.

15. The cannula of claim 14 wherein the first portion defines a first plane that is acutely angled at a first angle relative to the longitudinal axis and the second portion defines a second plane that is acutely angled at a second angle relative to the longitudinal axis and the second angle is greater than the first angle.

16. The cannula of claim 14 wherein the tip is chamfered.

17. The cannula of claim 15 wherein the tip is chamfered.

18. The cannula of claim 2 wherein the first notch is semicircular and the second notch is semicircular.

19. The cannula of claim 2 wherein the first notch is rectangular and the second notch is rectangular.

20. The cannula of claim 2 wherein the first notch is a ramped notch and the second notch is a ramped notch.

21. A cannula for intraocular surgery, the cannula comprising:
a cup;
a tube including a wall and having a tube outer diameter, the tube extending from the cup to a distal end and defining a longitudinal axis, the distal end including
a first portion and a second portion, the first portion including a chamfered tip and being narrower than the second portion; and
a gripping section formed in the wall, the gripping section having a diameter that is less than the tube outer diameter.

22. The cannula of claim 21 wherein the first portion defines a first plane that is acutely angled at a first angle relative to the longitudinal axis and the second portion defines a second plane that is acutely angled at a second angle relative to the longitudinal axis and the second angle is greater than the first angle.

23. The cannula of claim 21 wherein the gripping section includes a first notch formed in the wall and a second notch formed in the wall.

24. A cannula for intraocular surgery comprising:
a cup having an open first end and a second end; and
a hollow tube extending from the second end of the cup to a distal end, the hollow tube defining a longitudinal axis, and the distal end including a first portion and a second portion, the first portion including a tip and being narrower than the second portion.

25. The cannula of claim 24 wherein the first portion defines a first plane that is acutely angled at a first angle relative to the longitudinal axis and the second portion defines a second plane that is acutely angled at a second angle relative to the longitudinal axis and the second angle is greater than the first angle.

26. The cannula of claim 24 wherein the tip is chamfered.

27. The cannula of claim 25 wherein the tip is chamfered.
